(19) United States
(12) Patent Application Publication
(10) Pub. No.: US 2009/0042318 A1
(43) Pub. Date: Feb. 12, 2009

(54) METHOD FOR EFFECTIVE SEARCH FOR TARGET MOLECULE

(75) Inventors: Akito Tanaka, Ibaraki (JP); Akira Yamazaki, Osaka (JP); Masayuki Haramura, Kanagawa (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: REVERSE PROTEOMICS RESEARCH INSTITUTE CO., LTD., Tokyo (JP)

(21) Appl. No.: 11/814,986

(22) PCT Filed: Jan. 26, 2006
(86) PCT No.: PCT/JP2006/301715

§ 371 Date: Oct. 24, 2007

(30) Foreign Application Priority Data
Jan. 28, 2005 (JP) 2005-022119

(51) Int. Cl. G01N 33/545

(52) U.S. Cl. 436/531

(57) ABSTRACT

The present invention provides a solid phase carrier capable of adsorbing a highly hydrophobic target molecule, for example, a membrane associated protein, and a solid phase carrier optimized not only for a highly hydrophobic target molecule, but also for an optionally chosen target molecule. More specifically, the present invention provides a solid phase carrier having a ligand and a capping agent immobilized thereon, with the hydrophobic property of the surface thereof adjusted to enable the binding of the target molecule to the ligand, or to increase the amount of target molecule bound to the ligand; various methods using the solid phase carrier (for example, method of concentrating, isolating, or purifying a target molecule, a method of selectively adsorbing a particular target molecule to the solid phase carrier, or a method of analyzing the interaction between ligand and target molecule therefor); a production method for the solid phase carrier; and an improvement method for a solid phase carrier having a ligand and a capping agent immobilized thereon and the like.

### TECHNICAL FIELD

The present invention provides a novel solid phase carrier having a ligand and a capping agent immobilized thereon, various methods using the solid phase carrier, a method of producing the solid phase carrier, an improvement method for a solid phase carrier having a ligand and a capping agent immobilized thereon and the like.

### BACKGROUND ART

In recent years, there have been active attempts to search for a molecule having a specific interaction with a particular molecule, using a technique based on intermolecular interactions, or studies to extensively investigate such interactions. These are specifically represented by research in which a low-molecule-low-molecule, low-molecule-high-molecule, or high-molecule-high-molecule interaction is measured with one molecule immobilized on a solid phase carrier, or research in which a desired target (a molecule having a specific interaction with a molecule immobilized on a solid phase carrier) is purified based thereon. As examples of various techniques based on intermolecular interactions, 1) research into targets using affinity resin for the latter case, 2) methods applying surface plasmon resonance (SPR) or quartz crystal microbalance (QCM) for the former case are known well.

As examples of 1), the discovery of FKBP proteins, which bind to the immunosuppressant FK506 (FK506 binding proteins), using affinity resin, by Professor Schreiber in 1989 (discovery of FKBP12 as a protein that binds to FK506 in cells; Nature, Oct. 26, 1989, Vol. 341, pp. 758-760), the subsequently done discovery of calcineurin inhibitory action in the pharmacological action mechanism of FK506 by an FK506-FKBP complex (Cell, Aug. 23, 1991, Vol. 66, No. 4, pp. 807-815), the discovery of HDAC as a target protein for the anticancer agent Trapoxin (Science, Apr. 19, 1996, Vol. 272, pp. 408-411) and the like are known well. As examples of 2), BIACORE (trade name), which utilizes a gold thin film as the solid phase carrier and enables an extensive investigation of interactions of a compound, protein and the like with a protein and the like that specifically interact therewith, is known well.

However, to date, synthesis of affinity resin and the like has been performed by binding a ligand onto a solid phase carrier, and capping unreacted functional groups with, for example, acetyl groups and the like. In this case, if the solid phase carrier is hydrophilic, the surrounding of the ligand on the surface of the solid phase carrier obtained will become a hydrophilic environment. Therefore, for a combination of a target molecule and ligand whose binding is normally efficiently formed in a hydrophobic environment (for example, interaction between a membrane protein and a ligand, interaction between an enzyme having a highly oil-soluble substrate as the ligand and the ligand), the conditions in the above-described case are disadvantageous to the binding of the target molecule and the ligand; as a result, there have been failures to discover the desired protein. In fact, according to our survey, all discoveries of target proteins using affinity resin to date have been limited to cases where the target protein is a cytoplasmic protein; no membrane associated proteins have been discovered to date.

### DISCLOSURE OF THE INVENTION

It is an object of the present invention to provide a solid phase carrier capable of adsorbing a highly hydrophobic target molecule, for example, a membrane associated protein. It is another object of the present invention to provide a solid phase carrier optimized not only for a highly hydrophobic target molecule such as a membrane associated protein, but also for an optionally chosen target molecule.

The present inventors diligently investigated with the aim of solving the above-described problems, and succeeded in solving the above-described problems by intentionally reducing the amount of ligand immobilized to the solid phase carrier, capping an appropriately chosen hydrophobic substance over the remaining portion (that is, adjusting the binding density of the ligand and hydrophobic substance to the solid phase carrier) to artificially construct a hydrophobic environment on the solid phase surface. This technique is also considered to make it possible to efficiently perform an interaction between a ligand and target molecule as described above, which has conventionally been difficult even using measuring technologies such as a method applying surface plasmon resonance, based on the same concept. It has not been reported to date that the binding of a ligand to a target molecule is enabled, or the amount of ligand bound to a target molecule is increased, by adjusting the binding density of the ligand and the capping agent to the solid phase carrier, and/or choosing an appropriate capping agent, as far as the present inventors know.

Based on these findings, the present inventors developed the present invention. Accordingly, the present invention provides:

[1] A solid phase carrier having a ligand and a capping agent immobilized thereon,
wherein the hydrophobic property of the surface thereof is adjusted to allow the binding of a target molecule to the ligand, or to increase the amount of target molecule bound to the ligand.
[2] The solid phase carrier described in [1] above, wherein the adjustment of the hydrophobic property of the solid phase carrier surface is made by adjusting the binding density of the ligand and the capping agent.
[3] The solid phase carrier described in [1] above, wherein the adjustment of the hydrophobic property of the solid phase carrier surface is based on the selection of the capping agent.
[4] The solid phase carrier described in [1] above, wherein the adjustment of the hydrophobic property of the solid phase carrier surface is made by adjusting the binding density of the ligand and the capping agent, and selecting a capping agent.
[5] The solid phase carrier described in [1] above, wherein the target molecule is a protein.
[6] The solid phase carrier described in [5] above, wherein the protein is a membrane associated protein.
[7] The solid phase carrier described in [6] above, wherein the capping agent is a hydrophobic substance, the LOGP of the hydrophobic substance (excluding the functional group for immobilization) being not less than 3 when calculated as CLOGP.
[8] The solid phase carrier described in [7] above, wherein the hydrophobic substance is a compound represented by the following formula (I):

R₁—X (I)

[wherein R₁ is a hydrophobic portion selected from the group consisting of substituted or unsubstituted hydrocarbon groups and substituted or unsubstituted heterocyclic groups, X is a functional group for immobilization that forms a junction selected from the group consisting of —CO—NH—, —CO—O—, —NH—CH₂—, —NH═CH—, —CH₂—O—, —SO₂—NH—, —S—CH₂—, —S(O)—CH₂—, —SO₂—CH₂— and —SO₂—O— when bound to the functional group for immobilization on the solid phase carrier].
[9] The solid phase carrier described in [7] above, wherein the capping agent is stearic acid or a derivative thereof.
[10] The solid phase carrier described in [1] above, which is a resin, a metal or glass.
[11] The solid phase carrier described in [7] above, wherein the binding rate of ligand r_{L} (%) and the binding rate of capping agent r_{C} (%) meet the following conditional formula:

10≦r_{L}<90 and 10<r_{C}≦90
where r_{L}+r_{C}≦100.
   [12] The solid phase carrier described in [7] above, wherein the binding rate of ligand r_{L} (%) and the binding rate of capping agent r_{C} (%) meet the following conditional formula:

10≦r_{L}<70 and 30<r_{C}≦90
where r_{L}+r_{C}≦100.
   [13] A method of concentrating, isolating, or purifying a target molecule, comprising bringing a sample containing the target molecule into contact with a solid phase carrier having a ligand and a capping agent immobilized thereon, and recovering the target molecule adsorbed to the solid phase carrier, the hydrophobic property of the surface of the solid phase carrier being adjusted to enable the binding of the target molecule to the ligand, or to increase the amount of target molecule bound to the ligand.
   [14] A method of selectively adsorbing a particular target molecule to a solid phase carrier, comprising bringing a solid phase carrier having a ligand and a capping agent immobilized thereon into contact with a sample containing at least two kinds of target molecules having different hydrophobicities, allowing more selective adsorption of a target molecule having higher hydrophobicity, out of the at least two kinds of target molecules, to the solid phase carrier,
   the hydrophobic property of the surface of the solid phase carrier being adjusted to enable the binding of the target molecule having higher hydrophobicity to the ligand, or to increase the amount of binding of the target molecule having higher hydrophobicity to the ligand.
   [15] A method of analyzing an interaction between a ligand and a target molecule therefor, comprising binding to a solid phase carrier having a ligand and a capping agent immobilized thereon the target molecule therefor via the ligand, and measuring an interaction between the ligand and target molecule, the hydrophobic property of the surface of the solid phase carrier being adjusted to enable the binding of the target molecule to the ligand, or to increase the amount of the target molecule bound to the ligand.
   [16] A production method for a solid phase carrier having a ligand and a capping agent immobilized thereon, comprising immobilizing the ligand and the capping agent on the solid phase carrier while adjusting the hydrophobic property of the solid phase carrier surface to enable the binding of the target molecule to the ligand, or to increase the amount of target molecule bound to the ligand.
   [17] The production method described in [16] above, comprising the following steps (a) to (c):
   (a) a step for choosing a ligand to be immobilized on a solid phase carrier;
   (b) a step for determining the binding density of the ligand and the capping agent according to the kind of target molecule;
   (c) a step for immobilizing the ligand chosen in step (a) and a capping agent on the solid phase carrier, according to the binding density determined in step (b).
   [18] The production method described in [16] above, comprising the following steps (a) to (c):
   (a) a step for choosing a ligand to be immobilized on a solid phase carrier;
   (b) a step for choosing a capping agent to be immobilized on the solid phase carrier according to the kind of target molecule;
   (c) a step for immobilizing the ligand chosen in step (a) and the capping agent chosen in step (b) on the solid phase carrier.
   [19] The production method described in [16] above, comprising the following steps (a) to (d):
   (a) a step for choosing a ligand to be immobilized on a solid phase carrier;
   (b) a step for choosing a capping agent to be immobilized on the solid phase carrier according to the kind of target molecule;
   (c) a step for determining the binding density of the ligand and the capping agent according to the kind of target molecule and the hydrophobicity of the capping agent chosen in step (b);
   (d) a step for immobilizing the ligand chosen in step (a) and the capping agent chosen in step (b) on the solid phase carrier, according to the binding density determined in step (c).
   [20] An improvement method for a solid phase carrier having a ligand and a capping agent immobilized thereon, comprising evaluating a hydrophobic property of the solid phase carrier surface that enables the binding of the target molecule to the ligand, or increases the amount of target molecule bound to the ligand.
   [21] The improvement method described in [20] above, comprising the following steps (a) to (c):
   (a) a step for bringing a target molecule into contact with each of at least two kinds of solid phase carriers of different levels of the binding density of the ligand and the capping agent;
   (b) a step for determining and comparing the amounts of target molecule adsorbed to the at least two kinds of solid phase carriers;
   (c) a step for determining conditions with regard to the binding density of the ligand and the capping agent under which a larger amount of target molecule is adsorbed to the solid phase carrier, on the basis of the results of the comparison in (b).
   [22] The improvement method described in [20] above, comprising the following steps (a) to (c):
   (a) a step for bringing a target molecule into contact with each of at least two kinds of solid phase carriers having different kinds of capping agents;
   (b) a step for determining and comparing the amounts of target molecule adsorbed to the at least two kinds of solid phase carriers;
   (c) a step for determining conditions with regard to the kind of capping agent under which a larger amount of target molecule is adsorbed to the solid phase carrier, on the basis of the results of the comparison in (b).
   [23] The improvement method described in [20] above, comprising the following steps (a) to (c):
   (a) a step for bringing a target molecule into contact with each of at least two kinds of solid phase carriers having different levels of the binding density of the ligand and the capping agent and different kinds of capping agents;
   (b) a step for determining and comparing the amounts of target molecule adsorbed to the at least two kinds of solid phase carriers;
   (c) a step for determining conditions with regard to the binding density of the ligand and the capping agent and the kind of the capping agent under which a larger amount of target molecule is adsorbed to the solid phase carrier, on the basis of the results of the comparison in (b).

According to the present invention, it is possible to provide a solid phase carrier capable of adsorbing a highly hydrophobic target molecule, for example, a membrane associated protein. According to the present invention, it is also possible to provide a solid phase carrier optimized not only for a highly hydrophobic target molecule, but also for an optionally chosen target molecule. Such a solid phase carrier can be useful for column packing (for example, for chromatography), quartz crystal microbalances, arrays (for example, gene chips such as microarrays), surface plasmon resonance (SPR) chips and the like.

### BRIEF DESCRIPTION OF THE DRAWINGS

FIG. 1 shows the results of an analysis of the bindability of COX1 to ligand (ketoprofen)-immobilized resins (Toyopearl, AffiGel).

FIG. 2 shows the results of an analysis of the binding density of a ligand (ketoprofen) and a capping agent (stearic acid) to AffiGel, which has bindability to COX1.

FIG. 3 shows the results of an analysis of the binding density of a ligand (ketoprofen) and a capping agent (acetyl, stearic acid) to Toyopearl, which has bindability to COX1.

### BEST MODE FOR EMBODYING THE INVENTION

The present invention provides a solid phase carrier having a ligand and a capping agent immobilized thereon. The solid phase carrier of the present invention can be one having the hydrophobic property of the surface thereof adjusted to enable the binding of the target molecule to the ligand, or to increase the amount of target molecule bound to the ligand. The present inventors discovered that by adjusting as appropriate the hydrophobic property of the solid phase carrier surface, the binding of the target molecule to the ligand is enabled, or the amount of target molecule bound to the ligand is increased, and succeeded in developing a solid phase carrier having these features.

A ligand and target molecule are intended to mean a combination of members having a mutually specific interaction, with one member of the combination being immobilized to a solid phase carrier as the ligand, and the other serving as the target molecule; that is, their designations are interchangeable depending on which is to be immobilized on the solid phase carrier. There is not always only one kind of target molecule having a specific interaction with the ligand, and likewise there is not always only one kind of ligand having a specific interaction with the target molecule.

A “specific interaction” is a characteristic action to specifically recognize, and bind to, a particular ligand (a particular target molecule) only; the relation of a specific receptor to an agonist or an antagonist, the relation of an enzyme to a substrate, and, for example, the relation of an FK506-binding protein (target molecule) to FK506 (ligand), the relation of a steroid hormone receptor to a steroid hormone (e.g., dexamethasone and glucocorticoid receptor), the relation of HDAC to the anticancer agent trapoxin, and the like apply to a “specific interaction”.

The ligand to be immobilized on the solid phase carrier is not subject to limitation, and may be a low molecular compound or a high molecular compound, and is preferably a low molecular compound. Here, a low molecular compound is a compound having a molecular weight of less than about 1000; for example, an organic compound that can be used as a pharmaceutical and a derivative thereof, a naturally derived compound and a derivative thereof, a small nucleic acid molecule such as a protein binding site, present on an element such as a promoter/enhancer, peptides, saccharides (e.g., monosaccharides, disaccharides, oligosaccharides), metals and the like can be mentioned; the low molecular compound is preferably an organic compound that can be used as a pharmaceutical or a derivative thereof. A high molecular compound is a compound having a molecular weight of not less than about 1000; for example, proteins, nucleic acid molecules, polysaccharides and the like can be mentioned. A ligand is commercially available if it is a commonly known substance, or a ligand can be prepared in accordance with various reference documents. A novel substance can also be prepared as appropriate by utilizing various reactions in organic synthesis in common use in the art, or biological or gene engineering techniques.

The target molecule is not subject to limitation, and is the same as the ligand to be immobilized on the solid phase carrier; although it may be the above-described low molecular compound or high molecular compound, a high molecular compound is preferable. Particularly, a protein is preferable.

A capping agent refers to a substance, other than a ligand, that reacts with, and protects, a reactive functional group on a solid phase carrier surface to which no ligand binds. Although the capping agent is not subject to limitation, as long as it is a substance as described above, it is preferably a substance having a hydrophobicity different from that of the ligand. The capping agent used for immobilization on the solid phase carrier may be one kind or a mixture of two kinds or more. A capping agent is commercially available if it is a commonly known substance, or a capping agent can be prepared in accordance with various reference documents. A novel substance can also be prepared as appropriate by utilizing various reactions in organic synthesis in common use in the art.

The solid phase carrier on which a ligand and a capping agent are immobilized is not subject to limitation, but a solid phase carrier is chosen which is suitable for the intended use thereof, that is, to enable the binding of the target molecule to the ligand, or to increase the amount of target molecule bound to the ligand. As examples of the material, resins, glass, and metals (for example, gold, silver, iron, silicon) can be mentioned. These solid phase carriers may have any shape; for example, plates, beads, thin films, filaments, coils and the like can be mentioned. For example, beads consisting of a resin facilitate subsequent operation when packed in a column, and a metal thin film can be used as a carrier for BIACORE by surface plasmon resonance and the like. A glass plate is also preferable.

Although the solid phase carrier used in the present invention is not subject to limitation, as stated above, a synthetic resin is preferable. As examples of the synthetic resin, a sugar derivative resin, a methacrylate resin, a polystyrene resin, an acrylamide resin, an acrylic acid resin, a polyethylene resin, a polyisopropylene resin and the like can be mentioned. As examples of the sugar derivative resin, agarose derivatives and Sepharose derivatives can be mentioned. As examples of the monomer component of the methacrylate resin, one or two or more selected from the group consisting of methyl (meth)acrylate, ethyl (meth)acrylate, propyl (meth)acrylate, butyl (meth)acrylate, 2-ethylhexyl (meth)acrylate, lauryl (meth)acrylate, stearyl (meth)acrylate, cyclohexyl (meth)acrylate, 2-hydroxyethyl (meth)acrylate, 2-propyl (meth)acrylate, chloro-2-hydroxyethyl (meth)acrylate, diethylene glycol mono(meth)acrylate, methoxyethyl (meth)acrylate, glycidyl (meth)acrylate, dicyclopentanyl (meth)acrylate, dicyclopentenyl (meth)acrylate and isobornyl (meth)acrylate and the like can be mentioned.

An adjustment of the hydrophobic property of the solid phase carrier surface can be performed by, for example, adjusting the binding density of the ligand and the capping agent to the solid phase carrier. An adjustment of the binding density of the ligand and the capping agent can be performed by, for example, adjusting the binding rate of the ligand and the capping agent to reactive functional groups on the solid phase carrier surface. The binding rate of the ligand can be changed as appropriate according to the kinds of target molecule and solid phase carrier, and can be, for example, about 1 to 99%, preferably about 5 to 95%, more preferably about 10 to 90%, still more preferably about 20 to 80%, most preferably about 30 to 70%. By introducing a substance that confers the plurality of reactive functional groups required to immobilize the ligand and the capping agent to reactive functional groups on the solid phase carrier surface, it is also possible to improve the total density per se of the conjugate consisting of the ligand and the capping agent on the solid phase carrier surface.

An adjustment of the hydrophobic property of the solid phase carrier surface can also be performed by choosing a capping agent. For example, when the target molecule is a highly hydrophobic molecule, a highly hydrophobic capping agent is chosen; when the target molecule is a highly hydrophilic molecule, a highly hydrophilic capping agent is chosen.

Immobilization of a ligand and a capping agent to a solid phase carrier is performed by commonly known methods in common use in the art and methods consisting of an appropriate combination thereof; for example, immobilization by covalent bond or non-covalent bond, such as amide bond, Schiff base formation, C—C bond, ester bond, bond via a thiol group, hydrogen bond, and hydrophobicity interaction can be mentioned. All are performed using materials and reactions that are commonly known in the art. Each bond is performed by utilizing a reaction in common use in the art. As a convenient and accurate means, a method utilizing an amide bond formation reaction can be mentioned. This reaction can, for example, be performed according to “Peputido Gousei no Kiso to Jikken” (ISBN 4-621-02962-2, Maruzen, 1st edition issued in 1985). Regarding the reagents and solvents used in each reaction, those in common use in the art can be utilized, and are chosen as appropriate depending on the binding reaction employed. Whether or not the ligand and the capping agent have been immobilized on the solid phase carrier can be confirmed from the reaction rate measured by quantifying the amino groups on the solid phase carrier surface before and after the reaction (for example, ninhydrin test). The binding rate of the ligand and the capping agent can be adjusted by changing the relative amount of the reagent added. Specifically, for example, by adding a ligand to the reaction system at 0.5 equivalents to the functional groups on the solid phase carrier, and adding a reagent in excess to the extent that allows the ligand added to fully react with the solid phase carrier, a 50% ligand-immobilized carrier can be synthesized. Also, by quantifying the residual unreacted functional groups by an appropriate method, a ligand binding rate in more detail can be determined. On the other hand, a capping reaction can be achieved by reacting a capping reagent in excess with the residual functional groups.

In a mode of embodiment, the target molecule can be a highly hydrophobic molecule. As examples of the highly hydrophobic molecule, hydrophobic proteins such as intracellular hydrophobic proteins and membrane associated proteins can be mentioned.

As used herein, “an intracellular hydrophobic protein” means a highly hydrophobic protein present in cells. It is known that many of intracellular proteins not less than 3 to 40 kDa do not interact well with ligands unless brought into a somewhat hydrophobic environment. The solid phase carrier of the present invention is useful when the target molecule is a highly hydrophobic molecule, and is therefore capable of adsorbing a target molecule that requires a somewhat hydrophobic environment. As examples of such an intracellular hydrophobic protein, proteins present in organelle (for example, intranuclear proteins) and cytoplasmic proteins can be mentioned.

The “membrane associated protein” can be a protein partially embedded in a biomembrane such as the cell membrane, nuclear membrane, mitochondrial membrane, or endoplasmic reticular membrane, a protein passing the biomembrane, and a protein that transiently accumulates in the vicinity of the membrane (a protein that binds transiently and directly to the membrane, and a protein that transiently accumulates in the vicinity of the membrane by binding to another substance bound to the membrane (for example, protein or protein complex)). The solid phase carrier of the present invention is useful when the target molecule is a highly hydrophobic molecule; therefore, not only proteins partially embedded in a biomembrane and proteins passing the biomembrane, but also proteins that transiently accumulate in the vicinity of the membrane can be adsorbed, but because the solid phase carrier of the present invention is considered to be particularly useful when the hydrophobicity of the target molecule is higher, out of membrane associated proteins, proteins partially embedded in a biomembrane and proteins passing the biomembrane are more preferable. As examples of the membrane associated proteins, receptors, enzymes, channels, transporters, and pumps can be mentioned.

When the target molecule is a highly hydrophobic molecule, the solid phase carrier of the present invention can be one having a hydrophobic substance immobilized thereon as the capping agent. In this case, regarding the solid phase carrier of the present invention, the binding density of the capping agent on the solid phase carrier surface can be relatively higher than the binding density of the ligand as required.

As used herein, “a hydrophobic substance” refers to a substance that makes the environment around the ligand more hydrophobic when immobilized on the solid phase carrier along with the ligand, to enable the binding of a highly hydrophobic compound (target molecule) to the ligand, or to increase the amount of the compound bound to the ligand, other than the ligand. The degree of hydrophobicity can be generally expressed by hydrophobicity parameters; in the present invention, the hydrophobicity of “a hydrophobic substance” can be defined by a partition coefficient, specifically LOGP. In calculating LOGP, CLOGP (a predicted value obtained using a software program for estimating a hydrophobicity parameter of a compound by means of a computing machine; can be calculated using, for example, Corwin/Leo's program (CLOGP, Daylight Chemical Information System Co., Ltd.)) and the like are conveniently utilized, but the hydrophobicity parameter is not limited to CLOGP. The greater the CLOGP is, the higher the hydrophobicity is. In view of accomplishing the object of making the environment around the ligand more hydrophobic, and hence promoting the binding of a highly hydrophobic compound (target molecule) to the ligand, the LOGP of the hydrophobic substance of the present invention, when calculated as CLOGP, can be, for example, not less than 2.5, preferably not less than 3.5, more preferably not less than 4.5, still more preferably not less than 5.5, most preferably not less than 6.5 or 7. The LOGP of the hydrophobic substance of the present invention, when calculated as CLOGP, from the viewpoint of the ease of synthesis of the hydrophobic substance, can also be, for example, not more than 30, preferably not more than 20, more preferably not more than 15. More specifically, as such hydrophobic substances, saturated fatty acids (for example, arachic acid, stearic acid, myristic acid, palmitic acid, decanoic acid), unsaturated fatty acids (for example, arachidonic acid, linoleic acid, linolenic acid, oleic acid), surfactants (for example, NP-40), bile acids (for example, cholic acid, deoxycholic acid, chenodeoxycholic acid, lithocholic acid), or derivatives thereof (reactive derivatives) and the like can be mentioned. For example, the derivatives can be those derivatized with the substituent A described below.

However, of the portions contained in a hydrophobic substance, the functional group used for immobilization on the solid phase carrier often changes its structure after immobilization of the hydrophobic substance on the solid phase carrier, compared to the structure before immobilization. For example, provided that the hydrophobic substance has a hydrophobic portion (R₁) and —COOH, and the solid phase carrier has —NH₂, immobilization of the hydrophobic substance on the solid phase carrier can be accomplished by amide bond, but the hydrophobic substance after the immobilization will have —CO—, not —COOH. Hence, it is not the hydrophobic portion (R₁) and —COOH, but the hydrophobic portion (R₁) and —CO—, that contributes to the provision of a hydrophobic environment around the ligand on the solid phase carrier surface. Therefore, from the viewpoint of provision of a hydrophobic environment around the ligand on the solid phase carrier, i.e., the importance of the degree of hydrophobicity after immobilization on the solid phase carrier, it is appropriate that the hydrophobicity of the hydrophobic substance be expressed by a partial structure whose structure is preserved even after the immobilization on the solid phase carrier, rather than by the entire hydrophobic substance including the functional group used for the immobilization on the solid phase carrier. From this viewpoint, the hydrophobic substance can be represented by the hydrophobic portion R₁ and the functional group for immobilization X in the following formula (I):

R₁—X (formula I)

The hydrophobic portion (R₁) refers to the portion of a hydrophobic substance, resulting from removal of the functional group for immobilization, and responsible for the hydrophobicity of the hydrophobic substance. The LOGP of the hydrophobic portion (R₁), when calculated as CLOGP, can be, for example, not less than 3, preferably not less than 4, more preferably not less than 5, still more preferably not less than 6, most preferably not less than 7 or 8. The LOGP of the hydrophobic portion (R₁), when calculated as CLOGP, from the viewpoint of the ease of synthesis of the hydrophobic substance, can also be, for example, not more than 30, preferably not more than 20, more preferably not more than 15. Provided that the hydrophobic substance has a plurality of reactive functional groups, and that an optionally chosen one of these reactive functional groups is utilized at the time of immobilization on the solid phase carrier, the calculation of the LOGP of the hydrophobic substance (except the functional group for immobilization) must be based on the average value of LOGP of a partial structure lacking the optionally chosen reactive functional group.

The hydrophobic portion (R₁) is not subject to limitation, as long as it has a level of LOGP described above; more specifically, a substituted or unsubstituted hydrocarbon group and a substituted or unsubstituted heterocyclic group can be mentioned. The total carbon number in the substituted or unsubstituted hydrocarbon group and substituted or unsubstituted heterocyclic group can be, for example, not less than 9, preferably 9 to 99, more preferably 12 to 70, still more preferably 15 to 50.

As examples of the “substituted or unsubstituted hydrocarbon group” in R₁, substituted or unsubstituted linear hydrocarbon groups (for example, substituted or unsubstituted alkyl groups, substituted or unsubstituted alkenyl groups, substituted or unsubstituted alkynyl groups), substituted or unsubstituted cyclic hydrocarbon groups (for example, substituted or unsubstituted aryl groups, substituted or unsubstituted cycloalkyl groups, substituted or unsubstituted cycloalkenyl groups, substituted or unsubstituted cycloalkynyl groups) can be mentioned.

The “substituted or unsubstituted alkyl group” in R₁ is intended to mean an alkyl group substituted by one or more (for example, 1 to 5, preferably 1 to 3, more preferably 1 or 2) substituents selected from the group consisting of an aryl group optionally having a substituent, an alkoxy group optionally having a substituent, an amide group optionally having a substituent, a cycloalkyl group optionally having a substituent, a hetero aryl group optionally having a substituent, a carbonyl group optionally having a substituent, halogen atoms (for example, chlorine atom, iodine atom, bromine atom, fluorine atom), and a hydroxyl group (these substituents are hereinafter abbreviated “substituent A” as required), or an unsubstituted alkyl group.

As examples of the “alkyl group” of the “substituted or unsubstituted alkyl group” in R₁, nonanyl, decanyl, undecanyl, dodecanyl, tridecanyl, tetradecanyl, pentadecanyl, hexadecanyl, heptadecanyl, octadecanyl and the like can be mentioned.

As the “substituent” in the “aryl group optionally having a substituent”, alkyl groups (having the same definition as above), aryl groups having 6 to 10 carbon atoms (for example, phenyl, 1-naphthyl, 2-naphthyl and the like), aralkyl groups having 7 to 30 carbon atoms (for example, benzyl, phenethyl and the like), halogen atoms (for example, chlorine atom, iodine atom, bromine atom, fluorine atom), hydroxyl group, amino group, alkoxy groups having 1 to 30 carbon atoms (for example, methoxy, ethoxy, n-propoxy, isopropoxy, n-butoxy, isobutoxy, sec-butoxy, tert-butoxy), carboxyl group and the like can be mentioned. As examples of the “aryl group” in the “aryl group optionally having a substituent”, aryl groups having 6 to 10 carbon atoms, such as phenyl, 1-naphthyl, and 2-naphthyl can be mentioned.

As the “substituent” in the “alkoxy group optionally having a substituent”, aryl groups having 6 to 10 carbon atoms (for example, phenyl, 1-naphthyl, 2-naphthyl and the like), halogen atoms (for example, chlorine atom, iodine atom, bromine atom, fluorine atom), hydroxyl group, amino group, carboxyl group and the like can be mentioned. As examples of the “alkoxy group” in the “alkoxy group optionally having a substituent”, alkoxy groups having 1 to 30 carbon atoms, such as methoxy, ethoxy, n-propoxy, isopropoxy, n-butoxy, isobutoxy, sec-butoxy, and tert-butoxy can be mentioned.

As the “substituent” in the “amide group optionally having a substituent”, alkyl groups having 1 to 30 carbon atoms (for example, methyl, ethyl, propyl), aralkyl groups having 7 to 30 carbon atoms (for example, benzyl, phenethyl), halogen atoms (for example, chlorine atom, iodine atom, bromine atom, fluorine atom), hydroxyl group, amino groups, alkoxy groups having 1 to 30 carbon atoms (for example, methoxy, ethoxy, n-propoxy, isopropoxy, n-butoxy, isobutoxy, sec-butoxy, tert-butoxy), carboxyl groups and the like can be mentioned.

As the “substituent” in the “cycloalkyl group optionally having a substituent”, alkyl groups having 1 to 30 carbon atoms (for example, methyl, ethyl, propyl), aralkyl groups having 7 to 30 carbon atoms (for example, benzyl, phenethyl), halogen atoms (for example, chlorine atom, iodine atom, bromine atom, fluorine atom), hydroxyl group, amino group, alkoxy groups having 1 to 30 carbon atoms (for example, methoxy, ethoxy, n-propoxy, isopropoxy, n-butoxy, isobutoxy, sec-butoxy, tert-butoxy and the like can be mentioned), carboxyl group and the like can be mentioned. As the “cycloalkyl group” in the “cycloalkyl group optionally having a substituent”, cycloalkyl groups having 3 to 30 carbon atoms, such as cyclopropyl, cyclobutyl, cyclopentyl, cyclohexyl, and cyclooctyl can be mentioned.

As the “substituent” in the “hetero aryl group optionally having a substituent”, alkyl groups having 1 to 30 carbon atoms (for example, methyl, ethyl, propyl), aralkyl groups having 7 to 30 carbon atoms (for example, benzyl, phenethyl and the like), halogen atoms (for example, chlorine atom, iodine atom, bromine atom, fluorine atom), hydroxyl group, amino group, alkoxy groups having 1 to 30 carbon atoms (for example, methoxy, ethoxy, n-propoxy, isopropoxy, n-butoxy, isobutoxy, sec-butoxy, tert-butoxy), carboxyl group and the like can be mentioned. As the “hetero aryl group” in the “hetero aryl group optionally having a substituent”, thiazolyl, aminothiazolyl, furanyl, thiophenyl, pyrrolyl, indolyl and the like can be mentioned.

As the “substituent” in the “carbonyl group optionally having a substituent”, alkyl groups having 1 to 30 carbon atoms (for example, methyl, ethyl, propyl), aralkyl groups having 7 to 30 carbon atoms (for example, benzyl, phenethyl), halogen atoms (for example, chlorine atom, iodine atom, bromine atom, fluorine atom), hydroxyl group, amino group, alkoxy groups having 1 to 30 carbon atoms (for example, methoxy, ethoxy, n-propoxy, isopropoxy, n-butoxy, isobutoxy, sec-butoxy, tert-butoxy), carboxyl group and the like can be mentioned.

The “substituted or unsubstituted alkenyl group” in R₁ is intended to mean an alkenyl group substituted by substituent A or an unsubstituted alkenyl group. As examples of the “alkenyl group” of the “substituted or unsubstituted alkenyl group” in R₁, nonenyl, decenyl, undecenyl, dodecenyl, tridecenyl, tetradecenyl, pentadecenyl, hexadecenyl, heptadecenyl, octadecenyl and the like can be mentioned.

The “substituted or unsubstituted alkynyl group” in R₁ is intended to mean an alkynyl group substituted by substituent A or an unsubstituted alkynyl group. As examples of the “alkynyl group” of the “substituted or unsubstituted alkynyl group” in R₁, nonynyl, decynyl, undecynyl, dodecynyl, tridecynyl, tetradecynyl, pentadecynyl, hexadecynyl, heptadecynyl, octadecynyl and the like can be mentioned.

The “substituted or unsubstituted aryl group” in R₁ is intended to mean an aryl group substituted by substituent A or an unsubstituted aryl group. As examples of the “aryl group” of the “substituted or unsubstituted aryl group” in R₁, phenyl, 1-naphthyl, 2-naphthyl and the like can be mentioned.

The “substituted or unsubstituted cycloalkyl group” in R₁ is intended to mean a cycloalkyl group substituted by substituent A or an unsubstituted cycloalkyl group. As examples of the “cycloalkyl group” of the “substituted or unsubstituted cycloalkyl group” in R₁, cyclohexyl, cycloheptyl, cyclooctyl, and cyclononanyl can be mentioned. As the “cycloalkyl group” of the “substituted or unsubstituted cycloalkyl group” in R₁, groups resulting from condensation of a plurality of cycloalkyl groups (for example, a compound having a steroid backbone) are also included.

The “substituted or unsubstituted cycloalkenyl group” or “substituted or unsubstituted cycloalkynyl group” in R₁ is intended to mean a cycloalkenyl group or cycloalkynyl group substituted by substituent A, or an unsubstituted cycloalkenyl group or cycloalkynyl group.

The “substituted or unsubstituted heterocyclic group” in R₁ is intended to mean a heterocyclic group substituted by substituent A or an unsubstituted heterocyclic group. As examples of the “heterocyclic group” of the “substituted or unsubstituted heterocyclic group” in R₁, non-aromatic heterocyclic groups and aromatic heterocyclic groups can be mentioned.

The “non-aromatic heterocyclic group” of the “substituted or unsubstituted non-aromatic heterocyclic group” in R₁ is intended to mean a non-aromatic hetero group comprising carbon atoms and 1 to 3 hetero atoms selected from among nitrogen atoms, sulfur atoms and oxygen atoms; for example, pyrrolidinyl, piperidinyl, piperazinyl, pyrazolidinyl, morpholino and the like can be mentioned.

The “aromatic heterocyclic group” of the “substituted or unsubstituted aromatic heterocyclic group” in R₁ is intended to mean an aromatic hetero group comprising carbon atoms and 1 to 3 hetero atoms selected from among nitrogen atoms, sulfur atoms and oxygen atoms; for example, thienyl, furyl, pyridyl, quinolyl, isoquinolyl, pyrazinyl, pyrimidinyl, pyrrolyl, indolyl and the like can be mentioned.

The functional group for immobilization (X) refers to a functional group used to immobilize a capping agent (for example, hydrophobic substance) and ligand on a solid phase carrier. The functional group for immobilization is not subject to limitation, as long as it enables binding to the solid phase carrier, and it can be one that forms a junction selected from the group consisting of —CO—NH—, —CO—O—, —NH—CH₂—, —NH═CH—, —CH₂—O—, —SO₂—NH—, —S—CH₂—, —S(O)—CH₂—, —SO₂—CH₂— and —SO₂—O— when bound to the functional group for immobilization (Y) on the solid phase carrier. The functional group for immobilization (X) and the functional group for immobilization (Y) are not subject to limitation, as long as they form the above-described junction. For example, when the junction is —CO—NH—, the functional group for immobilization (X) may be —CO—OH and the functional group for immobilization (Y) may be —NH₂, or the functional group for immobilization (X) may be —NH₂ and the functional group for immobilization (Y) may be —COOH. Those skilled in the art are able to properly determine a combination of the functional groups for immobilization (X) and (Y) that forms the above-described junction.

As the hydrophobic substance, a substance that is hydrophobic as a whole despite the binding of a hydrophilic portion to the hydrophobic portion R₁ of the above-described formula I can also be used. Because such a substance has not only the hydrophobic portion but also the hydrophilic portion, and exhibits hydrophobicity as a whole, it can be considered to be capable of functioning as a virtual cell membrane. As such a substance, one wherein a saccharide (for example, monosaccharide, disaccharide, oligosaccharide) and a derivative thereof (for example, deoxy saccharide, uronic acid), a PEG derivative, a poly-OH derivative (for example, tartaric acid), as the hydrophilic portion, is bound to the hydrophobic portion R₁ can be mentioned.

Examples of hydrophobic substances that can be used in the present invention are shown below along with their molecular weights and the CLOGP of the entire structure and partial structure (hydrophobic portion). Odd numbers correspond to the entire structure of the hydrophobic substance, and subsequent even numbers correspond to the partial structure of the hydrophobic substance (hydrophobic portion). Calculations of CLOGP were performed using CLOGP version 4.0 (Daylight Company).

TABLE 1

| | STRUCTURE | MW | CLOGP |
|---|---|---|---|
| 1 | 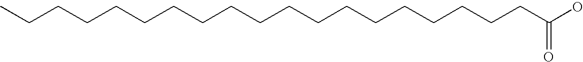 | 312.53 | 9.328 |
| 2 | 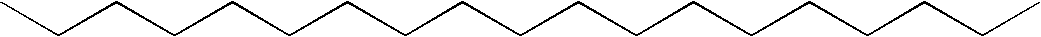 | 268.52 | 10.745 |
| 3 | 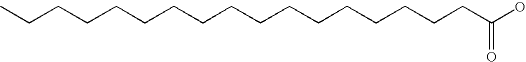 | 284.48 | 8.27 |
| 4 | 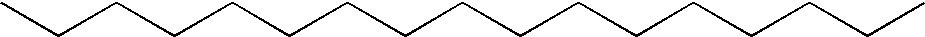 | 240.47 | 9.687 |
| 5 | 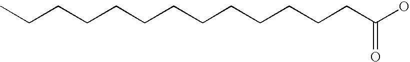 | 228.37 | 6.154 |
| 6 | 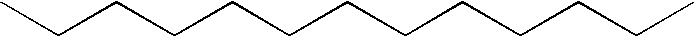 | 184.36 | 7.571 |
| 7 | 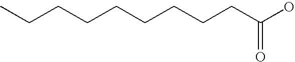 | 172.26 | 4.038 |
| 8 | 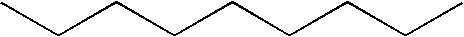 | 128.26 | 5.455 |
| 9 | 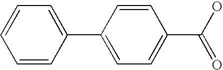 | 198.22 | 3.773 |
| 10 | 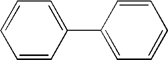 | 154.21 | 4.03 |
| 11 | 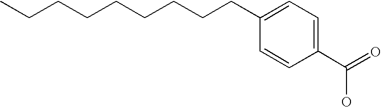 | 248.36 | 6.616 |
| 12 | 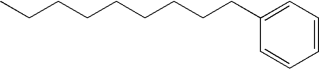 | 204.35 | 6.873 |
| 13 | 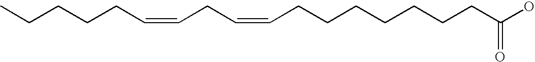 | 280.45 | 7.302 |
| 14 | 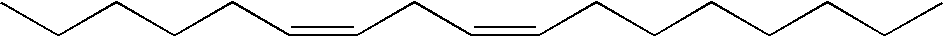 | 236.44 | 8.719 |
| 15 | 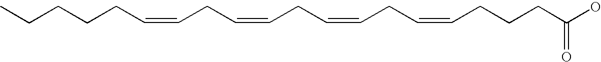 | 304.47 | 7.392 |
| 16 | 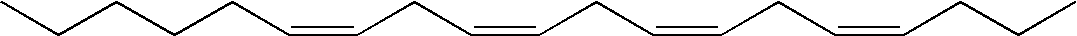 | 260.46 | 8.809 |
| 17 | 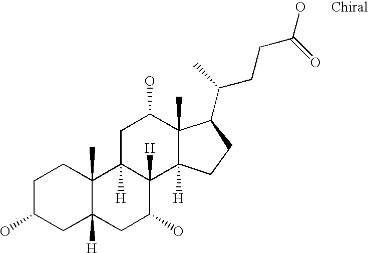 | 408.57 | 2.427 |
| 18 | 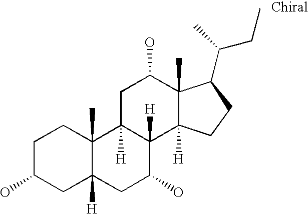 | 364.56 | 3.844 |
| 19 | 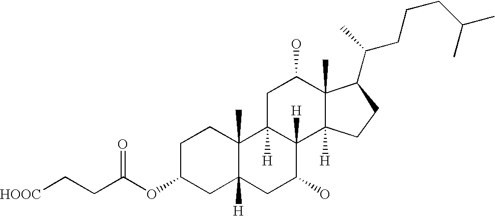 | 520.74 | 6.543 |
| 20 | 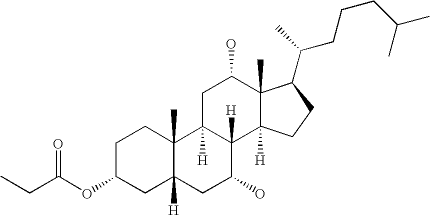 | 476.73 | 7.305 |
| 21 | 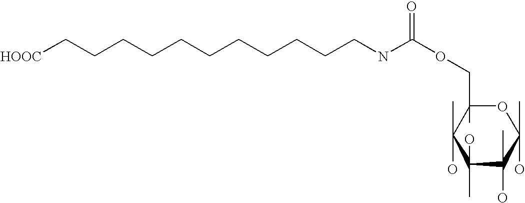 | 491.62 | 4.239 |
| 22 | 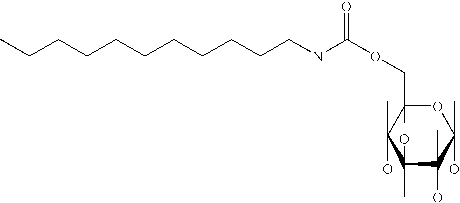 | 447.61 | 5.656 |
| 23 | 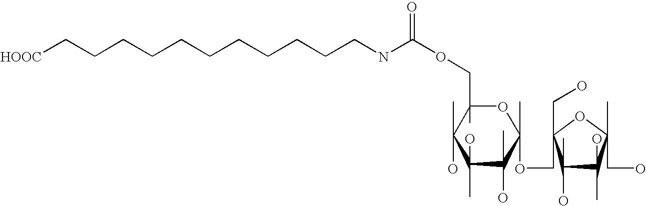 | 709.86 | 5.1 |
| 24 | 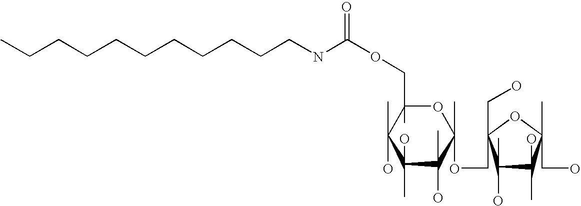 | 665.85 | 6.517 |
| 25 | 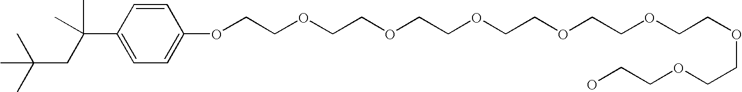 | 558.74 | 5.321 |
| 26 | 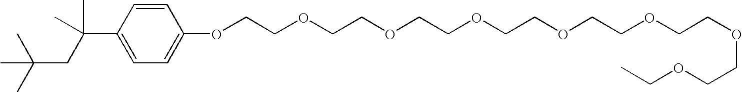 | 542.74 | 6.408 |
| 27 | 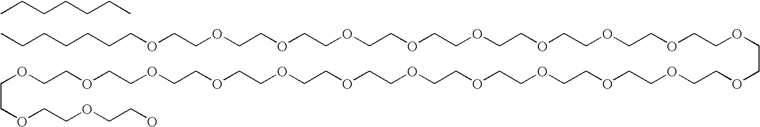 | 1243.6 | 6.554 |
| 28 | 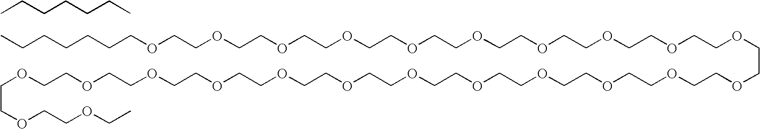 | 1227.6 | 7.641 |

The solid phase carrier of the present invention is more useful, for example, when the target molecule is a highly hydrophobic molecule, and the ligand is a weakly hydrophobic one. In a living body, a highly hydrophobic target molecule and a weakly hydrophobic ligand can form an interaction pair, and it is considered that a signal from the interaction pair can play a biologically important role, but such interacting pairs have been difficult to discover to date. It is believed that this is attributable, in part, to the fact that because it had conventionally been the mainstream to immobilize a ligand on a synthetic resin to maximum possible extent and cap the ligand with acetyl groups, it had been difficult to acquire a highly hydrophobic molecule as the target molecule, though it had been possible to acquire a weakly hydrophobic target molecule, when an insufficiently hydrophobic resin (in acquiring a highly hydrophobic compound such as a membrane associated protein, the hydrophobicity of the resin is often insufficient) is used as the solid phase carrier. However, because the present inventors found that for acquiring a highly hydrophobic molecule as the target molecule, it is important to change the hydrophobic property of the solid phase carrier surface, and that for changing the hydrophobic property of the solid phase carrier surface, for example, the binding density of the ligand and the capping agent (hydrophobic substance) may be adjusted, and/or an appropriate capping agent may be chosen, it has become possible to acquire a highly hydrophobic molecule as the target molecule, irrespective of the kind of synthetic resin.

As stated above, the solid phase carrier of the present invention exhibits its utility particularly when the target molecule is a highly hydrophobic molecule, and the ligand is a weakly hydrophobic one. The hydrophobicity of the ligand can be determined by a partition coefficient, specifically LOGP, as with the hydrophobicity of the hydrophobic substance and the like. When the target molecule is a highly hydrophobic molecule, the hydrophobicity of the ligand for which the solid phase carrier of the present invention exhibits its utility is not subject to limitation; the ligand hydrophobicity, when calculated as CLOGP, can be, for example, not more than 5, preferably not more than 4.5, more preferably not more than 4, still more preferably not more than 3.5, most preferably not more than 3, 2.5 or 2. The LOGP of the ligand, when calculated as CLOGP, can also be, for example, not less than 0.

However, for the same reason as that described above with respect to the hydrophobic substance, it is appropriate that the hydrophobicity of the ligand be expressed by the hydrophobicity of a partial structure whose structure is preserved even after immobilization on the solid phase carrier, rather than by the hydrophobicity of the entire ligand including the functional group used for the immobilization on the solid phase carrier (the functional group for immobilization). From this viewpoint, the LOGP of the ligand (except the functional group for immobilization), when calculated as CLOGP, is not subject to limitation, and it can be, for example, not more than 4.5, preferably not more than 4, more preferably not more than 3.5, still more preferably not more than 3, most preferably not more than 2.5, 2 or 1.5. The LOGP of the ligand (except the functional group for immobilization), when calculated as CLOGP, can also be, for example, not less than 0. Provided that the ligand has a plurality of reactive functional groups, and that an optionally chosen one of these reactive functional groups is used at the time of immobilization on the solid phase carrier, the calculation of the LOGP of the ligand (except the functional group for immobilization) must be based on the average value of LOGP of the partial structure lacking the optionally chosen reactive functional group.

When the target molecule is a highly hydrophobic molecule, the hydrophobic property of the surface of the solid phase carrier of the present invention can also be defined by the binding rate of ligand (r_{L}) and the binding rate of capping agent (r_{C}) as shown below. Here, the binding rate indicates the percentage of functional groups for immobilization to which the ligand or capping agent is bound, relative to all available functional groups for immobilization on the solid phase carrier surface. If the ligand and the capping agent bind to all available functional groups for immobilization on the solid phase carrier surface, r_{L}+rₕ=100 is assumed.

a≦r_{L}<b and c<r_{C}≦d
where r_{L}+r_{C}≦100.

When the target molecule is a highly hydrophobic molecule, a to d are not subject to limitation; for example, when a sugar derivative resin is used as the solid phase carrier, a can be, for example, 1%, preferably 5%, more preferably 10%, still more preferably 20%, most preferably 30%, 40% or 50%; b can be, for example, 99%, preferably 95%, more preferably 90%, still more preferably 80%, most preferably 70%; c can be, for example, 1%, preferably 5%, more preferably 10%, still more preferably 20%, most preferably 30%; d can be, for example, 99%, preferably 95%, more preferably 90%, still more preferably 80%, most preferably 70%, 60% or 50%. When a methacrylate resin is used as the solid phase carrier, a can be, for example, 1%, preferably 3%, more preferably 5%, still more preferably 7%, most preferably 10%; b can be, for example, 30%, preferably 25%, more preferably 20%; c can be, for example, 70%, preferably 75%, more preferably 80%; d can be, for example, 99%, preferably 97%, more preferably 9 5%, still more preferably 93%, most preferably 90%.

When the target molecule is a highly hydrophobic molecule, the hydrophobic property of the surface of the solid phase carrier of the present invention can also be defined by the following numerical formula (I).

CLOGP_{AVE}=(CLOGP_{L}×r_{L}+CLOGP_{C}×r_{C})/100 (numerical formula I)

where r_{L}+rₕ≦100
CLOGP_{AVE}: average CLOGP
CLOGP_{L}: CLOGP of ligand (except the functional group for immobilization)
CLOGP_{C}: CLOGP of capping agent (except the functional group for immobilization)
r_{L}: binding rate of ligand
r_{C}: binding rate of capping agent.

When the target molecule is a highly hydrophobic molecule, CLOGP_{AVE} in the numerical formula (I) above is not subject to limitation, and can be, for example, not less than 3, preferably not less than 3.5, more preferably not less than 4, still more preferably not less than 4.5, most preferably not less than 5, 5.5 or 6.

In another mode of embodiment, the target molecule can be a weakly hydrophobic molecule. As examples of the weakly hydrophobic molecule, hydrophilic proteins such as intracellular hydrophilic proteins and secretory proteins can be mentioned.

As used herein, an “intracellular hydrophilic protein” means a weakly hydrophobic protein present in cells, other than the above-described intracellular hydrophobic proteins. The solid phase carrier of the present invention is useful even when the target molecule is a weakly hydrophobic molecule. As examples of such an intracellular hydrophilic protein, proteins present in organelle (for example, intranuclear proteins) and cytoplasmic proteins can be mentioned.

A “secretory protein” means a protein secreted in the blood; for example, hormones, enzymes and the like can be mentioned.

When the target molecule is a weakly hydrophobic molecule, the solid phase carrier of the present invention can be one having a hydrophilic substance immobilized thereon as the capping agent. In this case, the binding density of the capping agent on the surface of the solid phase carrier of the present invention can be relatively higher than the binding density of the ligand as required.

As used herein, a “hydrophilic substance” refers to a substance that makes the environment around the ligand more hydrophilic when immobilized on the solid phase carrier along with the ligand, to enable the binding of a highly hydrophilic compound (target molecule) to the ligand, or to increase the amount of the compound bound to the ligand, other than the ligand. In view of accomplishing the object of making the environment around the ligand more hydrophilic, and hence promoting the binding of a highly hydrophilic compound (target molecule) to the ligand, the LOGP of the hydrophilic substance of the present invention, when calculated as CLOGP, can be, for example, less than 2.5, preferably less than 2, more preferably less than 1.5, still more preferably less than 1. The LOGP of the hydrophilic substance of the present invention, when calculated as CLOGP, can also be, for example, not less than −0.5. More specifically, as examples of such a hydrophilic substance, carboxylic acids having 1 to 6 carbon atoms (for example, acetic acid, butyric acid), saccharides (for example, monosaccharides, disaccharides, oligosaccharides), PEG derivatives, poly-OH derivatives (for example, tartaric acid) or derivatives thereof (reactive derivatives) and the like can be mentioned. For example, the derivatives can be those derivatized by the above-described substituent A.

A hydrophilic substance, from the same viewpoint as described above with respect to a hydrophobic substance, can also be expressed with the hydrophilic portion R₂ and the functional group for immobilization X using the following formula (I) (the functional group for immobilization X is the same as that described above):

R₂—X (formula I)

The hydrophilic portion (R₂) refers to the portion of a hydrophobic substance, resulting from removal of the functional group for immobilization, and responsible for the hydrophilicity of the hydrophilic substance. The LOGP of the hydrophilic portion (R₂), when calculated as CLOGP, can be, for example, less than 3, preferably less than 2.5, more preferably less than 2, still more preferably less than 1.5. The LOGP of the hydrophilic portion (R₂), when calculated as CLOGP, can also be, for example, not less than 0. Provided that the hydrophilic substance has a plurality of reactive functional groups, and that an optionally chosen one of these reactive functional groups is used at the time of immobilization on the solid phase carrier, the calculation of the LOGP of the hydrophilic substance (except the functional group for immobilization) must be based on the average value of LOGP of the partial structure lacking the optionally chosen reactive functional group.

The hydrophilic portion (R₂) is not subject to limitation, as long as it has the above-described LOGP; more specifically, substituted or unsubstituted hydrocarbon groups and substituted or unsubstituted heterocyclic groups can be mentioned. Although the total carbon number in the substituted or unsubstituted hydrocarbon group and substituted or unsubstituted heterocyclic group is never limited, it can be, for example, not more than 6, preferably not more than 4. A substituent for the hydrocarbon group and heterocyclic group can be chosen as appropriate from among, for example, substituent A, so that the hydrophobicity conditions are met.

The solid phase carrier of the present invention is more useful, for example, when the target molecule is a weakly hydrophobic molecule, and the ligand is a highly hydrophobic one. In the living body, a weakly hydrophobic target molecule and a highly hydrophobic ligand can form an interaction pair, and it is considered that a signal from the interaction pair can play a biologically important role, but such interacting pairs have been difficult to discover to date. It is believed that this is attributable, in part, to the fact that because it had conventionally been the mainstream to immobilize a ligand on a synthetic resin to maximum possible extent and cap the ligand with acetyl groups, it had been difficult to acquire a weakly hydrophobic target molecule when a resin whose hydrophobicity is not sufficiently low is used as the solid phase carrier. However, because the present inventors found that for acquiring a weakly hydrophobic molecule as the target molecule, it is important to change the hydrophobic property of the solid phase carrier surface, and that for changing the hydrophobic property of the solid phase carrier surface, for example, the binding density of the ligand and the capping agent (hydrophobic substance) may be adjusted, and/or an appropriate capping agent may be chosen, it has become possible to acquire a weakly hydrophobic molecule as the target molecule, irrespective of the kind of synthetic resin.

As stated above, the solid phase carrier of the present invention exhibits its utility particularly when the target molecule is a weakly hydrophobic molecule, and the ligand is a highly hydrophobic one. The hydrophobicity of the ligand can be defined by a partition coefficient, specifically LOGP, as with the hydrophobicity of the hydrophobic substance and the like. When the target molecule is a weakly hydrophobic molecule, the hydrophobicity of the ligand for which the solid phase carrier of the present invention exhibits its utility is not subject to limitation, and the ligand hydrophobicity, when calculated as CLOGP, can be, for example, not less than 2.5, preferably not less than 3.5, more preferably not less than 4.5, still more preferably not less than 5.5, most preferably not less than 6.5. The LOGP of the ligand, when calculated as CLOGP, can also be, for example, not more than 30, preferably not more than 20, more preferably not more than 15.

However, for the same reason as that described above with respect to the hydrophobic substance, it is appropriate that the hydrophobicity of the ligand be expressed by the hydrophobicity of a partial structure whose structure is preserved even after immobilization on the solid phase carrier, rather than by the hydrophobicity of the entire ligand including the functional group used for the immobilization on the solid phase carrier (the functional group for immobilization). From this viewpoint, the LOGP of the ligand (except the functional group for immobilization), when calculated as CLOGP, is not subject to limitation, and can be, for example, not less than 3, preferably not less than 4, more preferably not less than 5, still more preferably not less than 6, most preferably not less than 7. The LOGP of the ligand (except the functional group for immobilization), when calculated as CLOGP, can also be, for example, not more than 30, preferably not more than 20, more preferably not more than 15.

When the target molecule is a weakly hydrophobic molecule, the hydrophobic property of the surface of the solid phase carrier of the present invention can also be defined by the binding rate of ligand (r_{L}) and the binding rate of capping agent (r_{C}) as shown below.

a≦r_{L}<b and c<r_{C}≦d
where r_{L}+r_{C}≦100.

When the target molecule is a weakly hydrophobic molecule, a to d are not subject to limitation; for example, a can be, for example, 1%, preferably 5%, more preferably 10%, still more preferably 20%, most preferably 40%; b can be, for example, 99%, preferably 95%, more preferably 90%, still more preferably 80%, most preferably 60%; c can be, for example, 1%, preferably 5%, more preferably 10%, still more preferably 20%, most preferably 40%; d can be, for example, 99%, preferably 95%, more preferably 90%, still more preferably 80%, most preferably 60%.

When the target molecule is a weakly hydrophobic molecule, the hydrophobic property of the surface of the solid phase carrier of the present invention can also be defined by the following numerical formula (I).

CLOGP_{AVE}=(CLOGP_{L}×r_{L}+CLOGP_{C}×r_{C})/100 (numerical formula I)

where r_{L}+rₕ≦100

(CLOGP_{AVE}, CLOGP_{L}, CLOGP_{C}, r_{L}, and r_{C} are the same as those shown above, respectively)

When the target molecule is a weakly hydrophobic molecule, CLOGP_{AVE} in the numerical formula (I) above is not subject to limitation, and can be, for example, less than 3, preferably less than 2.5, more preferably less than 2, still more preferably less than 1.5.

The present invention also provides various methods using the solid phase carrier of the present invention. For example, the present invention provides a method of concentrating, isolating, or purifying a target molecule using the solid phase carrier of the present invention. The method of concentration, isolation, or purification of the present invention comprises, for example, bringing a sample containing the target molecule into contact with the solid phase carrier of the present invention, and recovering the target molecule adsorbed to the solid phase carrier. The sample is not subject to limitation; for example, when a column packed with the solid phase carrier of the present invention is used, the sample is preferably liquid. The method for bringing into contact with each other the sample and the solid phase carrier of the present invention is not subject to limitation, as long as the ligand and the target molecule can bind to each other by a specific interaction on the solid phase carrier of the present invention, if the target molecule is present in the sample. For example, when the solid phase carrier of the present invention is used as the packing in a column, the method can be conveniently performed by adding a liquefied sample to the column, and passing the sample through the column (column method). Conveniently, the method can be performed by mixing the solid phase carrier of the present invention and a sample for a specified time (batch method). The amount of sample applied to the column, flow rate, elution (recovery) treatment, mixing time and the like can be set on the basis of conditions in common use for affinity chromatography.

The present invention also provides a method of selectively adsorbing a particular target molecule to a solid phase carrier using the solid phase carrier of the present invention. The selective adsorption method of the present invention comprises, for example, bringing a solid phase carrier having a ligand and a capping agent immobilized thereon into contact with a sample containing at least two kinds of target molecules of different levels of hydrophobicity, thus allowing more selective adsorption of a target molecule having higher hydrophobicity, out of the at least two kinds of target molecules, to the solid phase carrier. The sample containing at least two kinds of target molecules of different levels of hydrophobicity, can be, for example, a sample containing both a highly hydrophobic target molecule (for example, membrane associated protein) and a weakly hydrophobic target molecule (for example, hydrophilic protein). By contacting the solid phase carrier of the present invention having the adjusted hydrophobic property of the surface thereof with a sample containing both a highly hydrophobic target molecule (for example, membrane associated protein) and a weakly hydrophobic target molecule (for example, hydrophilic protein), it is possible to selectively adsorb either the highly hydrophobic target molecule or the weakly hydrophobic target molecule by the solid phase carrier of the present invention. The selective adsorption method of the present invention may further comprise dissociating the adsorbed target molecule from the solid phase carrier of the present invention, and recovering the dissociated target molecule.

The present invention further provides a method of analyzing an interaction between a ligand and a target molecule therefor using the solid phase carrier of the present invention. The analytical method of the present invention comprises, for example, binding to a solid phase carrier having a ligand and a capping agent immobilized thereon a target molecule therefor via the ligand, and measuring an interaction between the ligand and target molecule (for example, mode of interaction, strength of interaction). A measurement of the interaction between the ligand and target molecule can be performed by a method known per se; for example, immunological methods (for example, immunoprecipitation, Western blotting), chromatography, mass spectrometry, amino acid sequencing, NMR, surface plasmon resonance, or a combination of these methods, and the like can be used.

The present invention also provides a method of producing the solid phase carrier of the present invention. The production method of the present invention comprises, for example, immobilizing a ligand and a capping agent to a solid phase carrier while adjusting the binding density of the ligand and the capping agent to enable the binding of the target molecule to the ligand, or to increase the amount of target molecule bound to the ligand.

In a mode of embodiment, the production method of the present invention comprises the following steps (a) to (c) (production method I):

(a) a step for choosing a ligand to be immobilized on a solid phase carrier;
(b) a step for determining the binding density of the ligand and the capping agent according to the kind of target molecule;
(c) a step for immobilizing the ligand chosen in step (a) and the capping agent chosen in step (b) on the solid phase carrier, according to the binding density determined in step (b).

In the step (a) of the production method I of the present invention, a ligand to be immobilized on a solid phase carrier is chosen. The solid phase carrier and ligand used are the same as those described above.

In the step (b) of the production method I of the present invention, the binding density of the ligand and the capping agent is determined according to the kind of target molecule. The binding density of the ligand and the capping agent can be determined from the viewpoint of the relative ratio of the ligand and the capping agent and/or the total density of the conjugate to the solid phase carrier consisting of the ligand and the capping agent. For example, depending on which of a highly hydrophobic compound or a weakly hydrophobic compound is to be used as the target molecule, the binding density can be changed as appropriate. In determining the binding density, the hydrophobicity of the ligand and/or the kind of solid phase carrier can also be taken into consideration as required. For example, in cases where a highly hydrophobic compound is intended as the target molecule with the hydrophobicity of the solid phase carrier used for immobilization being low, if the hydrophobicity of the capping agent is higher than that of the ligand, it can be judged that a larger amount of capping agent must be immobilized on the solid phase carrier.

In the step (c) of the production method I of the present invention, the ligand chosen in step (a) and a capping agent are immobilized on the solid phase carrier, according to the binding density determined in step (b). Immobilization of the ligand and the capping agent can be performed by a method known per se; for example, the above-described method can be used.

In another mode of embodiment, the production method of the present invention comprises the following steps (a) to (c) (production method II):

(a) a step for choosing a ligand to be immobilized on a solid phase carrier;
(b) a step for choosing a capping agent to be immobilized on the solid phase carrier according to the kind of target molecule;
(c) a step for immobilizing the ligand chosen in step (a) and the capping agent chosen in step (b) on the solid phase carrier.

The steps (a) and (c) of the production method II of the present invention can be performed in the same manner as the steps (a) and (c) of the production method I of the present invention.

In the step (b) of the production method II of the present invention, a capping agent to be immobilized on a solid phase carrier is chosen according to the kind of target molecule. For example, if a highly hydrophobic compound is chosen as the target molecule, an appropriate hydrophobic substance can be chosen as the capping agent, and if a weakly hydrophobic compound is chosen as the target molecule, an appropriate hydrophilic substance can be chosen as the capping agent. In choosing a capping agent, the hydrophobicity of the ligand and/or the kind of solid phase carrier can also be taken into consideration as required.

In still another mode of embodiment, the production method of the present invention can be one wherein the production methods I and II of the present invention are simultaneously performed. Such a production method comprises the following steps (a) to (d) (production method III):

(a) a step for choosing a ligand to be immobilized on a solid phase carrier;
(b) a step for choosing a capping agent to be immobilized on a solid phase carrier according to the kind of target molecule;
(c) a step for determining the binding density of the ligand and the capping agent according to the kind of target molecule and the hydrophobicity of the capping agent chosen in step (b);
(d) a step for immobilizing the ligand chosen in step (a) and the capping agent chosen in step (b) on the solid phase carrier, according to the binding density determined in step (c).

The present invention further provides an improvement method for a solid phase carrier having a ligand and a capping agent immobilized thereon. The improvement method of the present invention comprises, for example, evaluating a hydrophobic property of the solid phase carrier surface that enables the binding of the target molecule to the ligand, or increases the amount of target molecule bound to the ligand.

In a mode of embodiment, the improvement method of the present invention comprises the following steps (a) to (c) (improvement method I):

(a) a step for bringing a target molecule into contact with each of at least two kinds of solid phase carriers having different levels of the binding intensity of the ligand and the capping agent;
(b) a step for determining and comparing the amounts of target molecule adsorbed to the at least two kinds of solid phase carriers;
(c) a step for determining conditions with regard to the binding density of the ligand and the capping agent under which a larger amount of target molecule adsorbs to the solid phase carrier, on the basis of the results of the comparison in (b).

In the step (a) of the improvement method I of the present invention, at least two kinds of solid phase carriers having different levels of the binding density of the ligand and the capping agent are brought into contact with the target molecule. The at least two kinds of solid phase carriers having different levels of the binding density of the ligand and the capping agent can be ones that differ in terms of the relative ratio of the ligand and the capping agent and/or the total density of the conjugate to the solid phase carrier consisting of the ligand and the capping agent. Contact of the solid phase carrier and the target molecule can be performed by a method known per se; for example, the above-described method can be used. The target molecule may be a highly hydrophobic compound or a weakly hydrophobic compound. The target molecule may be brought into contact with the solid phase carrier in the form of a sample (for example, a biological sample) containing the molecule.

In the step (b) of the improvement method I of the present invention, the amounts of target molecule adsorbed to the at least two kinds of solid phase carriers having different levels of the binding density of the ligand and the capping agent are determined and compared. A determination of the amount of target molecule adsorbed to the solid phase carrier can be performed by a method known per se; for example, assay methods such as immunological methods (for example, immunoprecipitation, Western blotting), chromatography, mass spectrometry, and surface plasmon resonance can be used. Although a determination and comparison of the amount adsorbed may be performed for one kind of target molecule only, they may also be performed for a plurality of target molecules. For example, if both a highly hydrophobic compound and a weakly hydrophobic compound are found as target molecules, the amount adsorbed can be determined and compared for each of them.

In the step (c) of the improvement method I of the present invention, conditions with regard to the binding density of the ligand and the capping agent under which a larger amount of target molecule is adsorbed to the solid phase carrier are determined, on the basis of the results of the comparison in (b). According to this step, a preferable relative ratio of the ligand and the capping agent and/or a preferable total density of the conjugate to the solid phase carrier consisting of the ligand and the capping agent can be determined.

In another mode of embodiment, the improvement method of the present invention comprises the following steps (a) to (c) (improvement method II):

(a) a step for bringing a target molecule into contact with each of at least two kinds of solid phase carriers having different kinds of capping agents;
(b) a step for determining and comparing the amounts of target molecule adsorbed to the at least two kinds of solid phase carriers;
(c) a step for determining conditions with regard to the kind of capping agent under which a larger amount of target molecule is adsorbed to the solid phase carrier, on the basis of the results of the comparison in (b).

In the step (a) of the improvement method II of the present invention, the target molecule is brought into contact with each of at least two kinds of solid phase carriers having different kinds of capping agents. The step (a) of the improvement method II of the present invention can be performed in the same manner as the improvement method I of the present invention.

In the step (b) of the improvement method II of the present invention, the amounts of target molecule adsorbed to the at least two kinds of solid phase carriers having different kinds of capping agents are determined and compared. For example, if a highly hydrophobic compound is chosen as the target molecule, the solid phase carrier can be one having a hydrophobic substance of a different level of hydrophobicity immobilized thereon as the capping agent, and if a weakly hydrophobic compound is chosen as the target molecule, the solid phase carrier can be one having a hydrophilic substance of a different level of hydrophobicity immobilized thereon as the capping agent.

In the step (c) of the improvement method II of the present invention, conditions with regard to the kind of capping agent under which a larger amount of target molecule is adsorbed to the solid phase carrier are determined, on the basis of the results of the comparison in (b). For example, when the target molecule is a highly hydrophobic compound, which of a more highly hydrophobic substance and a more weakly hydrophobic substance is preferable can be determined, depending on the kind of ligand and solid phase carrier.

In another mode of embodiment, the improvement method of the present invention can be one wherein the improvement methods I and II of the present invention are simultaneously performed. Such an improvement method comprises the following steps (a) to (d) (improvement method III):

(a) a step for bringing a target molecule into contact with each of at least two kinds of solid phase carriers having different levels of the binding intensity of the ligand and the capping agent and different kinds of capping agents;
(b) a step for determining and comparing the amounts of target molecule adsorbed to the at least two kinds of solid phase carriers;
(c) a step for determining conditions with regard to the binding density of the ligand and the capping agent and the kind of the capping agent under which a larger amount of target molecule is adsorbed to the solid phase carrier, on the basis of the results of the comparison in (b).

The disclosures in all publications mentioned herein, including patents and patent application specifications, are incorporated by reference herein in the present invention to the extent that all of them have been given expressly.

The present invention is hereinafter described in more detail by means of the following Examples, which, however, are not to be construed as limiting the scope of the invention.

### EXAMPLES

### Production Example 1

### Preparation of Ligand and Capping Agent-Immobilized Solid Phase Carrier

### (1) Preparation of Ketoprofen and Stearic Acid-Immobilized Resin (AffiGel)

### Preparation of Affigel-50% Ketoprofen+50% Stearic Acid

After 1.2 ml (14.4 μmol) of Affi-Gel 102Gel (cat. 153-2401, BIO-RAD) was washed with 10 ml of DMF five times, 10 ml of dry DMF was added, and the mixture was stirred at room temperature for 1 hour. Ketoprofen (1.8 mg, 7.2 μmol), WSCD (cat. 1020, Peptide Institute, Inc.) (5.0 μl, 28.8 μmol), and HOBt (cat. 1022, Peptide Institute, Inc.) (3.9 mg, 28.8 μmol) were added, and the mixture was stirred at room temperature for one day and night. The resin was washed with DMF five times. As a result of a ninhydrin test, the ketoprofen retention rate was about 50%.

This resin was replaced with 10 ml of dry DMF, stearic acid (8.2 mg, 28.8 μmol), WSCD (6.1 μl, 34.6 μmol), and HOBt (4.7 mg, 34.6 μmol) were added, and the mixture was stirred at room temperature for one day and night. The resin was washed with DMF five times. After a portion of this sample was sampled, a ninhydrin test was performed; as a result, no residual amine was observed. After the resin was stirred in a 20% acetic anhydride solution in DMF for 30 minutes at room temperature and washed with DMF five times, it was washed with 20% ethanol solution, 10 ml×5, to yield the desired ketoprofen and stearic acid-immobilized resin (Affigel-50% ketoprofen+50% stearic acid).

By the same method, but using controlled amounts of reagents added, Affigel-60% ketoprofen+40% stearic acid, Affigel-70% ketoprofen+30% stearic acid, Affigel-80% ketoprofen+20% stearic acid, and Affigel-90% ketoprofen+10% stearic acid were obtained.

### Preparation of Affigel-100% Ketoprofen

After 1.0 ml (12 μmol) of Affi-Gel 102Gel (cat. 153-2401, BIO-RAD) was washed with 10 ml of DMF five times, 10 ml of dry DMF was added, and the mixture was stirred at room temperature for 1 hour. Ketoprofen (12.2 mg, 48 μmol), WSCD-HCl (11.0 mg, 57.6 μmol), and HOBt (7.8 mg, 57.6 μmol) were added, and the mixture was stirred at room temperature for one day and night. After the resin was washed with DMF five times, a ninhydrin test was performed; as a result, the desired compound was quantitatively obtained. After the resin was washed with DMF five times, the mixture was stirred in a 20% acetic anhydride solution in DMF at room temperature for 30 minutes. After the resin was washed with DMF five times, it was washed with 20% ethanol solution, 10 ml×5, to yield the desired Affigel-100% ketoprofen.

### Preparation of Affigel-Stearic Acid

After 1.2 ml (14.4 μmol) of Affi-Gel 102Gel (cat. 153-2401, BIO-RAD) was washed with 10 ml of DMF five times, 10 ml of dry DMF was added, and the mixture was stirred at room temperature for 1 hour. Stearic acid (16.4 mg, 57.6 μmol), WSCD (cat. 1020, Peptide Institute, Inc.) (12.1 μl, 57.6 μmol), and HOBt (cat. 1022, Peptide Institute, Inc.) (9.4 mg, 57.6 μmol) were added, and the mixture was stirred at room temperature for one day and night. After the resin was washed with DMF five times, a ninhydrin test was performed; as a result, the desired compound was quantitatively obtained. After the resin was washed with DMF five times, it was stirred in a 20% acetic anhydride solution in DMF at room temperature for 30 minutes. After the resin was washed with DMF five times, it was washed with 20% ethanol solution, 10 ml×5, to yield the desired Affigel-C18.

### (2) Preparation of Ketoprofen and Stearic Acid-Immobilized Resin (Toyopearl)

### Preparation of TOYO-20% Ketoprofen+80% C18

After 500 μl (50 μmol) of TOYOPEARL (AF-Amino-650M; cat. no 08039 TOSOH) was washed with 5 ml of DMF, it was washed with 5 ml of dichloromethane. 5 ml of dry dichloromethane was added, and the mixture was stirred at room temperature for 1 hour. Ketoprofen (2.54 mg, 10 μmol), PyBOP (26 mg, 50 μmol), and diisopropylethylamine (17.5 μl, 100 μmol) were added, and the mixture was stirred at room temperature for one day and night. The resin was washed with DMF five times. Subsequently, the resin was replaced with 5 ml of dry dichloromethane, stearic acid (28.4 mg, 100 μmol), PyBOP (104 mg, 200 μmol), and diisopropylethylamine (35 μl, 200 μmol) were added, and the mixture was stirred at room temperature for one day and night. After the resin was washed with DMF five times, a ninhydrin test was performed; as a result, a resin immobilized with residual amine by stearic acid was quantitatively obtained. The resin was stirred in a 20% acetic anhydride solution in DMF at room temperature for 30 minutes, and washed with DMF five times, after which it was washed with 20% ethanol solution, 10 ml×5, to yield the desired ketoprofen and stearic acid-immobilized resin (TOYO-20% ketoprofen+80% stearic acid).

By the same method, TOYO-10% ketoprofen+90% stearic acid, TOYO-30% ketoprofen+70% stearic acid, and TOYO-60% ketoprofen+40% stearic acid were synthesized.

### (3) Preparation of Ketoprofen and Acetyl-Immobilized Resin (Toyopearl)

### Preparation of TOYO-20% Ketoprofen+80% Acetyl

After 500 μl (50 μmol) of TOYOPEARL (AF-Amino-650M; cat. no 08039, TOSOH) was washed with 5 ml of DMF, it was washed with dichloromethane in the same manner. 5 ml of dry dichloromethane was added, and the mixture was stirred at room temperature for 1 hour. Ketoprofen (2.54 mg, 10 μmol), PyBOP (26 mg, 50 μmol), and diisopropylethylamine (17.5 μl, 100 μmol) were added, and the mixture was stirred at room temperature for one day and night. The resin was washed with DMF five times. Subsequently, the resin was stirred in 5 ml of a 20% acetic anhydride solution in DMF at room temperature for 30 minutes to protect the remaining amino groups with acetyl groups. The resin was washed with DMF five times, after which it was washed with 20% ethanol solution, 10 ml×5, to yield the desired ketoprofen and acetyl-immobilized resin (TOYO-20% ketoprofen+80% Ac).

By the same method, TOYO-10% ketoprofen+90% acetyl, TOYO-30% ketoprofen+70% acetyl, TOYO-60% ketoprofen+40% acetyl, and YOYO-100% ketoprofen were obtained.

### Example 1

### Analysis of Bindability of COX1 to Ketoprofen-Immobilized Resin

### (1) Preparation of COX1-Containing Lysate

10 μg of a commercially available COX1 (ovine) (cayman cat no. 60100) was added to 1 ml of a lysate (protein content: 1.5 mg/ml) of E. coli prepared by a conventional method using buffer A (Tris-HCl containing 0.5% Tween 20 and 300 μM DDC, pH 8.0), and this was used as the sample.

### (2) Analysis of Bindability of COX1 to Ketoprofen-Immobilized Resin

10 μl of the ketoprofen-immobilized resin and 1 ml of the lysate were gently shaken at 4° C. for one day and night. The resin was centrifuged at 1,2000×g, the supernatant was discarded, and the residual resin was washed with buffer A (1 ml) five times, after which 20 μl of SDS loading buffer (nakalai cat. No; 30566-22, 2-ME (2-mercaptoethanol) containing an electrophoretic sample buffer solution (2×)) was added, and the mixture was stirred at 25° C. for 10 minutes. The sample liquid thus obtained was separated using a commercially available SDS gel (BioRad ready Gel J, 10% SDS, cat. No; 161-J321), and the SDS gel was analyzed.

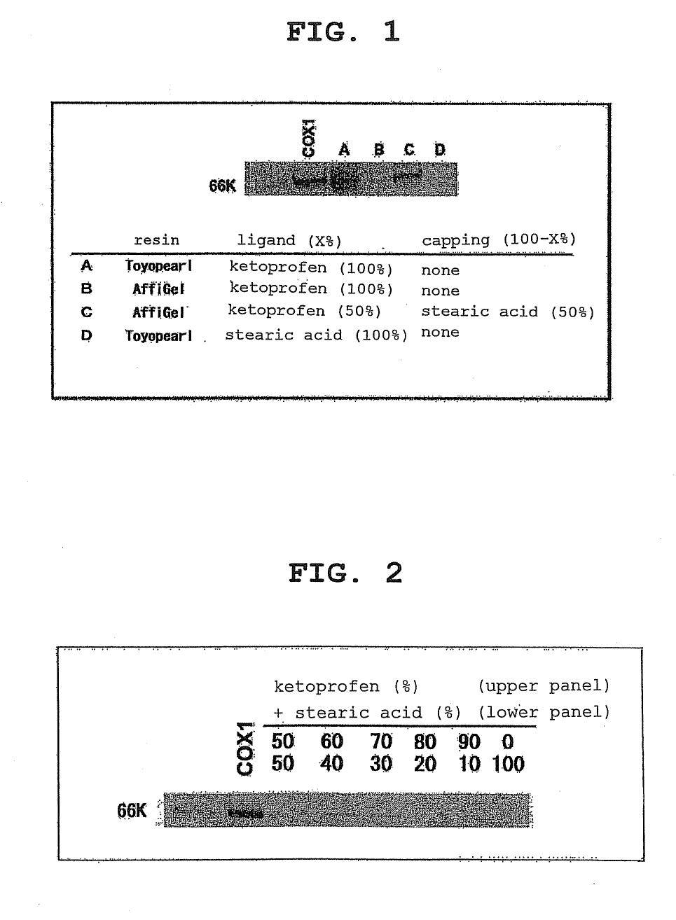

As a result, in a hydrophobic environment (Toyopearl), the ketoprofen-immobilized resin bound to COX1 (FIG. 1-A), but in a hydrophilic environment (AffiGel), the ketoprofen-immobilized resin was unable to bind to COX1 (FIG. 1-B). However, it was found that by subjecting the Affigel surface to stearic acid capping to provide a hydrophobic environment, AffiGel acquired bindability to COX1 (FIG. 1-C). The resin having stearic acid only immobilized thereon did not bind to COX1 (FIG. 1-D).

From the above, it was shown that by modifying the resin surface to provide a hydrophobic environment using stearic acid as the capping agent, a hydrophilic resin is made bindable to a membrane associated protein.

### Example 2

### Analysis of Binding Density of Ligand and Capping Agent to AffiGel, which has Bindability to COX1

From the results of Example 1, it was considered that for the binding of a target molecule to a ligand and a capping agent-immobilized resin, not only the kind of capping agent, but also the binding density of the ligand and the capping agent, may be important. Hence, again using ketoprofen as the ligand, stearic acid as the capping agent, and COX1 as the membrane associated protein, the binding density of ketoprofen and stearic acid to AffiGel, which has bindability to COX1, was analyzed. The COX1-containing lysate used was prepared in the same manner as Example 1, and the supplies of ketoprofen and stearic acid-immobilized AffiGel of different levels of the binding density used were prepared in accordance with Production Example 1.

As a result, it was demonstrated that by adjusting the binding density of ketoprofen and stearic acid to modify the hydrophobic property of the resin surface, COX1 bindability could be acquired (FIG. 2).

From the above, it was shown that by adjusting the binding density of the ligand and the capping agent to modify the hydrophobic property of the solid phase carrier surface, a weakly hydrophobic solid phase carrier acquired bindability to a highly hydrophobic target molecule.

### Example 3

### Analysis of Binding Density of Ligand and Capping Agent to Toyopearl, which has Bindability to COX1

The present inventors decided to further confirm the finding from the results of Examples 1 and 2 that for the binding of a target molecule to a ligand and a capping agent-immobilized resin, the kind of capping agent and the binding density of the ligand and the capping agent are important, using varied kinds of resins. Hence, using ketoprofen as the ligand, stearic acid or acetyl as the capping agent, and COX1 as the membrane associated protein, the binding density of the ligand and the capping agent to Toyopearl, which has COX1 bindability, was analyzed. The COX1-containing lysate used was prepared in the same manner as Example 1, and supplies of the ketoprofen and stearic acid or acetyl-immobilized Toyopearl of different levels of binding density were prepared in accordance with Production Example 1.

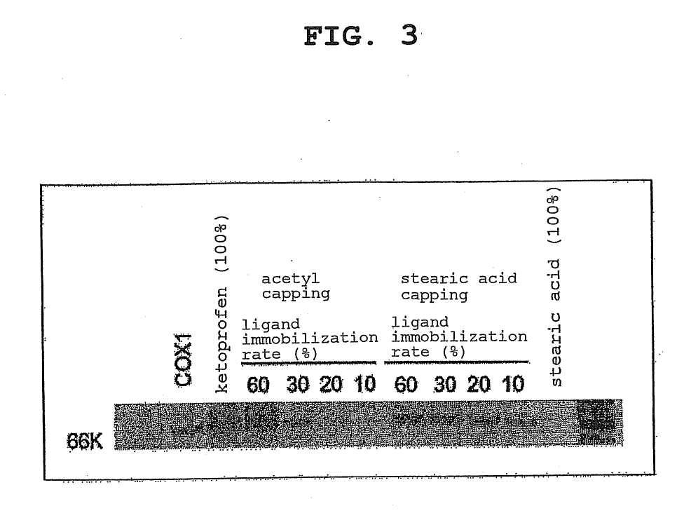

As a result, even Toyopearl lost the COX1 bindability when the binding density of the ligand was reduced and acetyl capping was performed to make the surface more hydrophilic (FIG. 3). By changing the capping agent to stearic acid, the COX1 bindability was retained (FIG. 3).

From the results of this Example and Examples 1 and 2, it was shown that to prepare a solid phase carrier having bindability to a target molecule, it is important to adjust the hydrophobic property of the solid phase carrier surface by choosing a kind of capping agent according to the kind of target molecule, and/or adjusting the binding density of the ligand and the capping agent.

### INDUSTRIAL APPLICABILITY

According to the present invention, it is possible to provide a solid phase carrier capable of adsorbing a highly hydrophobic target molecule, for example, a membrane associated protein. According to the present invention, it is also possible to provide a solid phase carrier optimized not only for a highly hydrophobic target molecule, but also for an optionally chosen target molecule. Such a solid phase carrier can be used for column packing (for example, for chromatography), quartz crystal microbalances, arrays (for example, gene chips such as microarrays), surface plasmon resonance (SPR) chips and the like.

This application is based on a patent application No. 2005-22119 filed in Japan on Jan. 28, 2005, the contents of which are incorporated in full herein by this reference.

1. A solid phase carrier having a ligand and a capping agent immobilized thereon,
wherein the hydrophobic property of the surface thereof is adjusted to allow the binding of a target molecule to the ligand, or to increase the amount of target molecule bound to the ligand.

2. The solid phase carrier of claim 1, wherein the adjustment of the hydrophobic property of the solid phase carrier surface is made by adjusting the binding density of the ligand and the capping agent.

3. The solid phase carrier of claim 1, wherein the adjustment of the hydrophobic property of the solid phase carrier surface is made based on the selection of the capping agent.

4. The solid phase carrier of claim 1, wherein the adjustment of the hydrophobic property of the solid phase carrier surface is made by adjusting the binding density of the ligand and the capping agent, and selecting a capping agent.

5. The solid phase carrier of claim 1, wherein the target molecule is a protein.

6. The solid phase carrier of claim 5, wherein the protein is a membrane associated protein.

7. The solid phase carrier of claim 6, wherein the capping agent is a hydrophobic substance, the LOGP of the hydrophobic substance (excluding the functional group for immobilization) being not less than 3 when calculated as CLOGP.

8. The solid phase carrier of claim 7, wherein the hydrophobic substance is a compound represented by the following formula (I):
R₁—X (I)
[wherein R₁ is a hydrophobic portion selected from the group consisting of substituted or unsubstituted hydrocarbon groups and substituted or unsubstituted heterocyclic groups, X is a functional group for immobilization that forms a junction selected from the group consisting of —CO—NH—, —CO—O—, —NH—CH₂—, —NH═CH—, —CH₂—O—, —SO₂—NH—, —S—CH₂—, —S(O)—CH₂—, —SO₂—CH₂— and —SO₂—O— when bound to the functional group for immobilization on the solid phase carrier].

9. The solid phase carrier of claim 7, wherein the capping agent is stearic acid or a derivative thereof.

10. The solid phase carrier of claim 1, which is a resin, a metal or glass.

11. The solid phase carrier of claim 7, wherein the binding rate of ligand r_{L} (%) and the binding rate of capping agent r_{C} (%) meet the following conditional formula:
10≦r_{L}<90 and 10<r_{C}≦90
where r_{L}+r_{C}≦100.

12. The solid phase carrier of claim 7, wherein the binding rate of ligand r_{L} (%) and the binding rate of capping agent r_{C} (%) meet the following conditional formula:
10≦r_{L}<70 and 30<r_{C}≦90
where r_{L}+r_{C}≦100.

13. A method of concentrating, isolating, or purifying a target molecule, comprising bringing a sample containing the target molecule into contact with a solid phase carrier having a ligand and a capping agent immobilized thereon, and recovering the target molecule adsorbed to the solid phase carrier, the hydrophobic property of the surface of the solid phase carrier being adjusted to enable the binding of the target molecule to the ligand, or to increase the amount of target molecule bound to the ligand.

14. A method of selectively adsorbing a particular target molecule to a solid phase carrier, comprising bringing a solid phase carrier having a ligand and a capping agent immobilized thereon into contact with a sample containing at least two kinds of target molecules having different hydrophobicities, allowing more selective adsorption of a target molecule having higher hydrophobicity, out of the at least two kinds of target molecules, to the solid phase carrier,
the hydrophobic property of the surface of the solid phase carrier being adjusted to enable the binding of the target molecule having higher hydrophobicity to the ligand, or to increase the amount of binding of the target molecule having higher hydrophobicity to the ligand.

15. A method of analyzing an interaction between a ligand and a target molecule therefor, comprising binding, to a solid phase carrier having a ligand and a capping agent immobilized thereon the target molecule therefor via the ligand, and measuring an interaction between the ligand and the target molecule,
the hydrophobic property of the surface of the solid phase carrier being adjusted to enable the binding of the target molecule to the ligand, or to increase the amount of the target molecule bound to the ligand.

16. A production method for a solid phase carrier having a ligand and a capping agent immobilized thereon, comprising immobilizing the ligand and the capping agent on the solid phase carrier while adjusting the hydrophobic property of the solid phase carrier surface to enable the binding of the target molecule to the ligand, or to increase the amount of target molecule bound to the ligand.

17. The production method of claim 16, comprising the following steps (a) to (c):
(a) a step for choosing a ligand to be immobilized on a solid phase carrier;
(b) a step for determining the binding density of the ligand and the capping agent according to the kind of target molecule;
(c) a step for immobilizing the ligand chosen in step (a) and a capping agent on the solid phase carrier, according to the binding density determined in step (b).

18. The production method of claim 16, comprising the following steps (a) to (c):
(a) a step for choosing a ligand to be immobilized on a solid phase carrier;
(b) a step for choosing a capping agent to be immobilized on the solid phase carrier according to the kind of target molecule;
(c) a step for immobilizing the ligand chosen in step (a) and the capping agent chosen in step (b) on the solid phase carrier.

19. The production method of claim 16, comprising the following steps (a) to (d):
(a) a step for choosing a ligand to be immobilized on a solid phase carrier;
(b) a step for choosing a capping agent to be immobilized on the solid phase carrier according to the kind of target molecule;
(c) a step for determining the binding density of the ligand and the capping agent according to the kind of target molecule and the hydrophobicity of the capping agent chosen in step (b);
(d) a step for immobilizing the ligand chosen in step (a) and the capping agent chosen in step (b) on the solid phase carrier, according to the binding density determined in step (c).

20. An improvement method for a solid phase carrier having a ligand and a capping agent immobilized thereon, comprising evaluating a hydrophobic property of the solid phase carrier surface that enables the binding of the target molecule to the ligand, or increases the amount of target molecule bound to the ligand.

21. The improvement method of claim 20, comprising the following steps (a) to (c):
(a) a step for bringing a target molecule into contact with each of at least two kinds of solid phase carriers of different levels of the binding density of the ligand and the capping agent;
(b) a step for determining and comparing the amounts of target molecule adsorbed to the at least two kinds of solid phase carriers;
(c) a step for determining conditions with regard to the binding density of the ligand and the capping agent under which a larger amount of target molecule is adsorbed to the solid phase carrier, on the basis of the results of the comparison in (b).

22. The improvement method of claim 20, comprising the following steps (a) to (c):
(a) a step for bringing a target molecule into contact with each of at least two kinds of solid phase carriers having different kinds of capping agents;
(b) a step for determining and comparing the amounts of target molecule adsorbed to the at least two kinds of solid phase carriers;
(c) a step for determining conditions with regard to the kind of capping agent under which a larger amount of target molecule is adsorbed to the solid phase carrier, on the basis of the results of the comparison in (b).

23. The improvement method of claim 20, comprising the following steps (a) to (c):
(a) a step for bringing a target molecule into contact with each of at least two kinds of solid phase carriers having different levels of the binding density of the ligand and the capping agent and different kinds of capping agents;
(b) a step for determining and comparing the amounts of target molecule adsorbed to the at least two kinds of solid phase carriers;
(c) a step for determining conditions with regard to the binding density of the ligand and the capping agent and the kind of the capping agent under which a larger amount of target molecule is adsorbed to the solid phase carrier, on the basis of the results of the comparison in (b).
